(19) United States
(12) Patent Application Publication
(10) Pub. No.: US 2010/0112662 A1
(43) Pub. Date: May 6, 2010

(54) MICROORGANISM FOR PRODUCING RECOMBINANT PIG LIVER ESTERASE

(75) Inventors: Uwe T. Bornscheuer, Greifswald (DE); Dominique Boettcher, Sponholz (DE); Elke Bruesehaber, Luebeck (DE); Kai Doderer, Rodgau (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK DEGUSSA GmbH, Essen (DE)

(21) Appl. No.: 12/305,834

(22) PCT Filed: Jun. 11, 2007
(86) PCT No.: PCT/EP2007/055692

§ 371 Date: Jan. 8, 2010

(30) Foreign Application Priority Data
Jul. 6, 2006 (DE) 102006031600.2

(51) Int. Cl. C12N 9/16; C12N 1/21

(52) U.S. Cl. 435/196; 435/252.33

(57) ABSTRACT

The invention relates to a method for the processing of reaction solutions, containing whole-cell catalysts, an aqueous component and an organic component, wherein the organic component contains the product to be enriched, with the following steps: a) adjustment of the pH value to less than 4; b) filtration of the reaction solution in the presence of a filter aid, preferably in the reaction solution; c) optionally: further enrichment and/or purification of the product contained in the organic component.

The present invention relates to a microorganism which comprises at least one copy of a polynucleic acid sequence which is foreign to the host and which encodes a protein having an enzymic activity, and comprises a chaperone system which assists the expression of the protein in the form of an active enzyme, and to a method for producing a protein having esterase activity using such a microorganism.

Lipases and esterases are suitable as efficient biocatalysts for the preparation of a large number of optically active compounds. Whereas, however, a whole series of lipases—especially of microbial origin—are commercially available, only very few esterases are available for use in a racemate resolution in industrial quantities [Bornscheuer, U. T. and Kazlauskas R. J., Hydrolases in Organic Synthesis (2005), 2^{nd} ed, Wiley-VCH, Weinheim].

In this connection there is particular interest in pig liver esterase because of its interesting catalytic properties in organic synthesis [Faber, K., Biotransformations in Organic Chemistry (2004), 5^{th} ed. Springer, Berlin; Jones, J. B. Pure Appl. Chem., (1990), 62, 1445-1448, Jones et. al. Can. J. Chem. (1985), 63, 452-456; Lam, L. K. P. et. al., J. Org. Chem. (1986), 51, 2047-2050].

Although it has been possible to show that stereoselective conversion of substrates is possible in some cases with esterase extracts from pig liver tissue, the use of such extracts is associated with a number of disadvantages, however. Besides variations in the esterase content between different batches, the presence of other hydrolases is to be regarded in particular as problematic in relation to stereoselectivities (Seebach, D. et. al, 25 Chimia (1986), 40, 315-318). There is in addition the problem that the conventional extracts take the form of a plurality of isoenzymes (Farb, D., et. al, Arch. Biochem. Biophys. (1980) 203, 214-226) which in some cases differ considerably in their substrate specificity. Heymann, E. and Junge, W. (Eur. J. Biochem. (1979), 95, 509-518; Eur. J. Biochem. (1979), 95, 519-525) achieved an elaborate electrophoretic separation making it possible to isolate fractions which preferentially cleave butyrylcholine, proline β-naphthylamide and methyl butyrate. In contrast thereto, other investigations (e.g. Lam, L. K. P., et. al, J. Am. Chem. Soc. (1988) 110, 4409-4411) merely show differences in the activity, but not in the specificity of individual fractions.

For this reason, there is a need for biotechnologically produced pig liver esterases with a defined composition.

Although cloning of putative pig liver esterase genes has been known for some time (Takahashi, T, et. al., J. Biol. Chem. (1989), 264, 11565-11571; FEBS Lett. (1991), 280, 297-300; FEBS Lett. (1991), 293, 37-41; David, L. et. al, Eur. J. Biochem. (1998) 257, 142-148), the functional, recombinant expression of an active pig liver esterase has been achieved to date, despite considerable efforts owing to the existing demand for this enzyme, only in Pichia pastoris (Lange, S. et al., ChemBioChem (2001), 2, 576-582). The productivities achieved in this case are very low, at 0.5 U/ml of culture supernatant after fermentation for hours, and are thus unsatisfactory for commercial production of the pig liver esterase. It would additionally be desirable to use Escherichia coli as expression system, because this expression system is associated with the advantages mentioned below.

Systems based on Escherichia coli for heterologous expression have many advantages over other expression hosts (Makrides, S C, Microbiol Rev (1996), 60, 512-582) for producing large quantities of recombinant proteins. The principal advantages are the rapid growth of Escherichia coli cells, the high content of heterologously expressed protein and the detailed knowledge of the biology, the metabolism and the genetics of these organisms. It is nevertheless not possible for every gene to be produced heterologously, actively and with good productivities in Escherichia coli. This may be due to the unique and unpredictable properties of the gene, the stability and the translational efficiency of the messenger RNA (mRNA), the degradation of the recombinant protein by proteases intrinsic to the cell or fundamental differences in the codon usage of the expression host and of the foreign gene (Jana, S. et al., Appl. Microbiol. Biotechnol. (2005), 67, 289-298).

Expression in bacterial hosts may in addition generally have some fundamental disadvantages, especially if the heterologously expressed protein(s) is/are derived from eukaryotic sources. In many cases, the recombinant protein is then inappropriately folded and thus insoluble and inactive. The reasons for this are based on the fact that, owing to the biology of Escherichia coli, no post-translational modifications corresponding to the eukaryotic systems are carried out, such as, for example, glycosylations and others (e.g. Jana, S. et al., Appl. Microbiol. Biotechnol. (2005), 67, 289-298 and all references therein). It is likewise impossible for the recombinant target protein to be secreted into the medium. This is necessary for the functional folding of many proteins (e.g. Jana, S. et al., Appl. Microbiol. Biotechnol. (2005), 67, 289-298 and all references therein). The ability of E. coli cells to form disulphide bridges in the target protein is likewise very limited. Since efficient and correct formation of disulphide bridges is, however, essential for functional folding of many proteins, this is a very important point.

Investigations of esterase extracts from pig liver show that the individual isoforms of the proteins are glycosylated. This likewise applies to the isoform produced recombinantly in Pichia pastoris (Lange, S. et. al., ChemBioChem (2001), 2, 576-582).

E. coli expression systems described in the literature for the heterologous expression of proteins have been tested by comparison with the present invention. The attempt to express pig liver esterase in Escherichia coli BL21Star™ (DE3) led to overexpression of the protein in the form of inclusion bodies. However, no pig liver esterase activity was detectable in the E. coli crude cell extract, although the E. coli strain BL21Star™ (DE3) used lacks two important proteases which are responsible for the degradation of expressed proteins (see comparative example 1). The lack of such proteases usually means a marked reduction in the degradation of the heterologously expressed proteins.

E. coli Rosetta (DE3) was used as further E. coli expression strain. Six tRNAs for codons which are represented very rarely in wild-type E. coli strains have been added to this expression host. This usually leads to improvement in the expression of foreign proteins, especially of eukaryotic origin [Novy, R. et. al., in Novations (2001), 12, 1-3]. Use of this expression strain also led only to expression in the form of inclusion bodies. No pig liver esterase activity was detectable in the supernatant after cell disruption (see comparative example 2).

The formation of disulphide bridges in a heterologously expressed target protein can be improved through the use of an E. coli strain which has mutations in the thioredoxin reductase gene and glutathione reductase gene and thus improves the conditions for the formation of disulphide bridges in the cytosol of E. coli [Besette, P. H. et. al., Proc. Natl. Acad. Sci. USA (1999), 96, 13703-13708)]. E. coli Origami (DE3) has this modification and was used for the expression of the pig liver esterase. The expression detected in this case took place exclusively in the form of inclusion bodies, and no pig liver esterase activity was detectable in the crude cell extract (see comparative example 3).

It is possible in many cases to achieve functional expression by reducing the inducer concentration (Thomas, J G, Protein Expression and Purif. (1997), 11, 289-296). This was carried out using the E. coli Rosetta-gami (DE3) strain. This strain combines all the properties of the three E. coli strains described above, and the level of expression of a protein can be adapted by varying the inducer concentration (use of IPTG as inducer). Even with this procedure and by reducing the IPTG concentration, most of the expression of the heterologous protein took place in the form of inclusion bodies and, after disruption of the E. coli cells, only a low, commercially unattractive pig liver esterase activity was detectable (see comparative example 4).

The literature likewise describes additions to the medium during expression in E. coli. Addition of up to 3% (v/v) ethanol to the medium induces the formation of chaperones belonging to E. coli., enzymes which serve as folding aids and usually assist correct folding (Thomas, J G, Protein Expression and Purif (1997), 11, 289-296). Expression of pig liver esterase in E. coli Origami (DE3) with addition of 3% (v/v) ethanol to the medium likewise led to no detectable active expression of the esterase in E. coli (see comparative example 5), but only to inclusion bodies.

It can be stated in summary that to date no functional expression of pig liver esterase in Escherichia coli has yet been reported. However, in order to utilize the advantages described above for the Escherichia coli expression system, it was an object of the present invention to find a system and a method for the functional expression of a desired heterologous enzyme in an Escherichia coli host.

The object is achieved by a microorganism comprising at least one copy of a polynucleic acid sequence which is foreign to the host (heterologous) and which encodes a protein having an enzymic activity, and a chaperone system which assists the functional expression of the protein in the form of an active enzyme, and a method for producing a functional enzyme using such a microorganism.

A host organism according to the invention which is preferred and particularly suitable is an E. coli strain whose expression properties are known. It is particularly preferred for the E. coli strain to be able to carry out certain post-translational modifications on the expressed protein, e.g. the formation of disulphide bridges or, where appropriate, also glycosylations. It is likewise preferred for the strain to provide a possibility for regulating expression and/or for proteins belonging to the host and reducing the expression yield (e.g. proteases) to be deleted.

A preferred enzyme which can be expressed with the aid of such a microorganism is an esterase, preferably an esterase from mammals, particularly preferably a porcine esterase which is naturally expressed in the pig liver. Such an esterase preferably has a stereoselective catalytic activity. A particularly preferred esterase is one encoded by the cDNA sequence SEQ ID No. 1 or fragments thereof, or by a sequence which is homologous to this sequence or fragments thereof. It may be sufficient for the present invention if fragments of SEQ ID NO. 1 or of a sequence homologous thereto are expressed and lead to amino acid sequences which possess a catalytic activity which corresponds to the desired activity. A preferred esterase thus has an amino acid sequence SEQ ID No. 5 or a homologous sequence, or fragments thereof which possess a catalytic activity.

A homologous sequence in connection with the present invention means, at the polynucleic acid level (i.e. at the DNA/RNA level), a sequence which, owing to the degeneracy of the genetic code, leads to the same amino acid sequence which is also encoded by SEQ ID No. 1 (this corresponds to 100% homology), in particular a sequence which is adapted for example to the species-specific codon usage of the host, or a polynucleic acid sequence which encodes a homologous protein, it being necessary in this case also to take account of the degeneracy of the genetic code. A sequence is homologous at the protein level (a homologous protein) according to the present invention if the amino acid sequence of the protein has been modified by comparison with SEQ ID No. 5 in such a way that a catalytic esterase activity still exists. The amino acid sequence is preferably modified by comparison with SEQ ID No. 5 in such a way that at least 70%, preferably at least 80%, further preferably at least 90% and particularly preferably at least 95% of the amino acids are identical to the respective amino acids in the same position in the sequential arrangement. It is additionally preferred for the amino acids which have been modified by comparison with SEQ ID No. 5 to be “homologous amino acids”, i.e. in each case an amino acid which resembles the amino acid present at the corresponding position in SEQ ID No. 5 in charge, steric extent and polarity. Examples of a homologous amino acid exchange are the exchange of alanine, serine or threonine for one another, aspartate and glutamate for one another, asparagine and glutamine for one another, arginine and lysine for one another, isoleucine, leucine, methionine and valine for one another, and phenylalanine, tyrosine and tryptophan for one another, without obligatory restriction thereto. Enzymes which are likewise to be regarded as homologues of the enzyme described herein are those which have in their catalytic region a homology which complies with the above definition, but differ in the N-terminal or C-terminal region from SEQ ID No. 5. Possible examples thereof are in particular splice variants of the present enzyme which, however, have the same or a very similar activity as the enzyme with SEQ ID No. 5, or else tissue-specific variants of the enzyme.

The sequence which codes for the desired protein to be expressed is preferably introduced with the aid of a plasmid into the host cell. For this purpose, the sequence is preferably provided in the form of cDNA, is inserted by customary methods familiar to the skilled person into a suitable vector and is introduced into the target cell. The methods of plasmid construction and transformation of the target cells are in no way limiting for the present invention. It is possible to use all methods known to the skilled person and leading to a suitable expression host which can express the desired sequence in a functional manner.

The choice of the vector used for constructing the plasmid is not limiting either, as long as it is possible to obtain a plasmid which enables expression, preferably inducible expression, in the chosen host. A preferred plasmid can be expressed in E. coli, preferably in the E. coli strain Origami (DE3) (obtainable from Novagen, Madison, Wis., USA). A particularly preferred vector for the plasmid construction for expressing the desired protein is the vector pET15b (Novagen, Madison, Wis., USA) which has suitable cloning cleavage sites for inserting desired sequences. The plasmid pET15b_mPLE constructed from this vector and the preferred esterase sequence represents a plasmid to be used particularly preferably according to the invention in a suitable host organism. This complete construct is depicted in SEQ ID No. 2.

According to the present invention, the host organism comprises a chaperone system which is suitable and able to assist the folding of the expressed heterologous protein to give a functional enzyme with catalytic activity. Chaperones are so-called “folding helper proteins” which are “of assistance” in the correct three-dimensional arrangement of an amino acid sequence to give the “finished” protein, specifically both during expression of the protein and in the correction of “disarrangements”, e.g. after the denaturation of proteins. Chaperones are also known as “heat shock proteins” because there is a distinct enhancement of expression thereof in cells after brief exposure to elevated temperatures. Various chaperone systems have been disclosed, and one of the best-investigated systems is the GroEL/GroES system, a bacterial chaperone system in which the two factors GroEL and GroES cooperate closely.

A chaperone system which is preferably used according to the invention is one which brings about the correct folding, leading to an enzymic activity, of heterologous proteins, in particular of mammalian proteins, it being possible to dispense with a post-translational modification, which normally takes place where appropriate in the original cell, of the protein. A chaperone system which is preferably used according to the present invention includes at least GroEL and GroES, it also being possible for other chaperones to be present, but the chaperones Dnak, DnaJ and GrpE are particularly preferably not present. In a preferred embodiment, the chaperone system employed according to the invention can be induced by an initiating stimulus which can easily be applied and which otherwise has no adverse effect on the host cells. Although chaperones can be induced naturally by a heat shock, in most cases this also has an effect on the other conditions of the cells and may lead for example to extensive denaturation of proteins. It is therefore preferred to bring about the induction of the chaperone system for example by adding an inducing substance. Such inducible chaperone systems are known to the skilled person and are commercially available on the market, with polynucleic acid sequences which encode the desired chaperones to be used being provided on plasmids. Induction of the expression of these sequences is achieved by a sequence which is located upstream on the plasmid and which, after addition of an inducing substance, regulates an increase in the expression of the sequences following it. One supplier of chaperone plasmid sets is for example TAKARA BIO Inc., Otsu, Japan. However, it is possible to use any other plasmids from various suppliers which provide chaperone systems which are suitable for use according to the present invention.

The desired chaperone system is preferably likewise introduced into a suitable host organism, e.g. by transformation or transfection of the host cell. It is thus preferred to prepare a transgenic cell which is able, through the introduction of suitable polynucleic acid sequences which code for the desired chaperone system, to provide the desired chaperone system, preferably after induction. However, the information for the desired chaperone system may also already be present in the host cell in its own genome, so that the selection of suitable host cells which provide an appropriate chaperone system is also suitable for the invention. However, the chaperone system should preferably be inducible by a stimulus which is not otherwise disadvantageous. This is the case primarily when plasmids which include the inducible chaperone system as coding sequences are used.

A particularly preferred plasmid to be introduced for the purposes of the present invention into a suitable host is the plasmid pGro7 which is obtainable from TAKARA BIO Inc., Otsu, Japan. However, all other commercially available plasmids which provide the GroEL/GroES system as inducible system are likewise to be regarded as preferred.

A host organism which is rendered, through the introduction of the heterologous polynucleic acid sequence(s), specifically at least the sequence for the enzyme to be expressed and where appropriate, or preferably, the sequences for the chaperone system, capable of expressing the desired protein as functional enzyme with a catalytic activity can be used to synthesize the enzyme or at least a catalytically active fragment thereof and to produce the latter in economically worthwhile quantities.

The present invention therefore likewise relates to a method for producing a catalytically active protein (fragment thereof), preferably a protein with esterase activity as described in detail above, where the protein is expressed by a microorganism into which a polynucleic acid sequence coding for the heterologous protein has been introduced, and which has a, preferably inducible, chaperone system which makes it possible for the enzyme to be provided in its functional form.

All organisms to be used are of course to be cultured and stimulated to expression under culturing conditions which allow growth and expression of the heterologous protein. Suitable culturing conditions are known to every person skilled in the area of microbiology and molecular biology and are generally notified by the distributors of the organisms or of the chaperone or expression systems.

A particularly preferred embodiment of the method is a method for producing the pig liver esterase with SEQ ID No. 5 with coexpression of the chaperones GroEL and GroES in an E. coli Origami (DE3) strain. Surprisingly, expression of the active enzyme was achieved in the presence of these two folding helper proteins, although other alternative chaperone systems such as, for example, the chaperones belonging to E. coli and induced by ethanol addition (see comparative example 5), or other coexpressed chaperones such as DnaK, DnaJ and GrpE, do not lead to success (see comparative example 6).

It is surprising to the skilled person that equivalent coexpression of the two chaperone systems Dnak, DnaJ, GrpE and GroEL, GroES together with the pig liver esterase in E. coli Origami (DE3) leads only to expression in the form of inclusion bodies and not to a detectable activity in E. coli crude cell extract (see comparative example 6). It is therefore preferred in every case for the GroEL/GroES chaperone system to be the system which is preferably induced/expressed even if other chaperone systems are present at the same time in the host organism.

Functional expression of eukaryotic proteins in E. coli represents a great challenge, especially if the proteins undergo post-translational glycosylation. In the case of the recombinant expression of pig liver esterase in E. coli, the use of the specific GroEL, GroES chaperone system apparently compensates for the lack of post-translational glycosylation.

### FIGURES

FIG. 1 shows the plasmid map of the plasmid pET15b_mPLE

Some exemplary embodiments are given below to illustrate the invention, but they are not to be regarded as restrictive.

### EXAMPLES

General information, and microorganisms, media, vectors and oligonucleotides used:

The Escherichia coli strains E. coli One Shot® TOP10 competent cells (Invitrogen, Carlsbad, Calif., USA) [F-mcrA D(mrr-hsdRMSmcrBC) (F801acZDM15) DlacX74 recA1 deoR araD139 D(ara-leu)7697 galU galK rpsL (StrR) endA1 nupG] or DHSα [supE44ΔlacU169 (Φ80lacZΔM15) hsdR17 recA1 endA1 gyrA96 thi-lrelA1] were used to maintain and replicate the plasmids. The E. coli strains Rosetta (DE3) [F-ompT hsdSB(rB-mB-) gal dcm (DE3) pRARE2 (CamR)], Origami (DE3) [A(ara-leu)7697 AlacX74 AphoA PvuII phoR araD139 ahpC galE galK rpsL F' [lac+lacIq pro] (DE3) gor522::Tn10 trxB (KanR, StrR, TetR)4], Rosetta-gami B (DE3) [F-ompT hsdSB(rB- mB-) gal dcm lacY1 aphC (DE3) gor522::Tn10 trxB pRARE2 (CamR, KanR, TetR)], all from Novagen (Madison, Wis., USA), and BL21Star™ (DE3) [F-ompT hsdSB(rB- mB-) gal dcm rne131 (DE3)] are used for the expression experiments.

The E. coli cells are cultured in Luria Bertani (LB) medium [yeast extract (5 g L⁻¹), peptone (10 g L⁻¹), NaCl (10 g L⁻¹)], to which the necessary antibiotics are added, at various temperatures (20-37° C.).

Primer 1 (SEQ ID NO. 3):
5′-GCCATATGGGGCAGCCAGCCTCGCCGCCTG-3′
Primer 2 (SEQ ID NO. 4):
5′-GATCCTCGAGTCACTTTATCTTGGGTGGC-3′

The plasmids pG-KJE8, pGro7, pKJE7 were purchased from TAKARA BIO Inc., Otsu, Shiga, Japan, in the form of a chaperone plasmid set. The plasmid pGro7 has a p15A origin of replication and a chloramphenicol resistance. The genes which code for the chaperones GroEL and GroES are located behind an arabinose promoter. Addition of a suitable quantity of arabinose is followed by expression of the folding helpers GroEL and GroES [Nishihara, K.; et al., Appl. Environ. Microbiol. (1998), 64, 1694-1699].

The vector pET15b was purchased from Novagen. The vector pET15b has a ColE1 origin of replication and an ampicillin resistance. The vector possesses a so-called multiple cloning site into which the gene to be expressed is cloned. These genes are then under the control of the strong T7 promoter [The pET System Manual, 11^{th} edition, Novagen (TB055)].

### General Information: DNA Recombination and Transformation

Unless mentioned otherwise, standard methods according to Sambrook, J. and Russell, D. W., Molecular Cloning, A Laboratory Manual, (2001), 3^{rd} ed., Cold Spring Harbour, N.Y., were used.

A QiAprep spin miniprep kit, a plasmid midi kit or a QIAquick gel extraction kit (Qiagen, Hilden, Germany) was used for plasmid and DNA extraction. The restriction enzymes employed were used in accordance with the respective manufacturer's information. The DNA sequencing was carried out by MWG-Biotech (Ebersberg, Germany. A standard protocol of Chung, C. T., et al. (1989) Proc. Natl. Acad. Sci. USA. 86, 2172-2175, was used for the preparation and transformation of competent E. coli cells.

### SDS Polyacrylamide Gel Electrophoresis and Zymogram

20 μl of commercially available pig liver carboxylesterase purchased from Sigma-Aldrich (100 U according to pNPA assay), dissolved in 2 ml as control and 20 μl of the cell lysate of the E. coli cultures were mixed with 10 μl of a 2×SDS sample buffer. After the solution had been heated at 95° C. for 5 min, the proteins were separated on a 12.5% polyacrylamide gel with 4% stacking gel. The samples were stained with Coomassie Brilliant Blue R250 to detect proteins.

For the esterase activity determination, the proteins fractionated in the polyacrylamide gel were renatured in a Triton X-100 solution (0.5% in 0.1 M Tris/HCl pH 7.5) for 1 hour. The gel was then mixed with a 1:1 mixture of solution A (20 mg of a-naphthyl acetate dissolved in 5 ml of acetone and subsequent addition of 50 ml of 0.1 M Tris/HCl pH 7.5) and solution B (50 mg Fast Red TR salt dissolved in 50 ml of 0.1 M Tris/HCl, pH 7.5). In the presence of hydrolytic lipase or esterase activity, a red α-naphthyl form of the Fast Red is formed (Krebsfänger, N., et al., (1998) Enzyme Microb. Technol., 22, 641-646).

### Determination of the Esterase Activity

The esterase activity was determined by photometry in a sodium phosphate buffer (50 mM, pH 7.5). The substrate used was p-nitrophenyl acetate (10 mM dissolved in DMSO). The liberated amount of p-nitrophenol was determined at 410 nm (e=12.36×10³ M⁻¹cm⁻¹) at room temperature. The enzymic activity was additionally determined with variation of the pH. A unit U is defined as an esterase activity with which 1 μmol of p-nitrophenol is liberated per minute under assay conditions.

Determination of the Protein Content in the E. coli Crude Cell Extract

The protein assay from Bio-Rad was used for Bradford determination of the protein concentrations in solution. The assay is based on the use of the dye Coomassie Brilliant Blue G-250 which binds with high specificity to proteins. In an acidic solution of Coomassie Brilliant Blue G-250 bound to proteins there is a shift in the absorption maximum of the unbound dye from 465 nm to 595 nm [Bradford, M. M., Anal. Biochem. (1976), 72, 248-254]. Protein quantities in the range 1-20 μg can be determined with this method.

Cell Disruption with Ultrasound 1 g of wet cell mass is resuspended in 10 ml of sodium phosphate buffer (50 mM, pH 7.5) and treated with ultrasound on ice for 3×1 min with a one minute pause in each case (80 W, pulses 35% s⁻¹). Centrifugation is then carried out at 3300 g and 4° C. for 20 min in order to remove cell detritus. The clear supernatant is used for further experiments.

### Construction of the Expression Vector

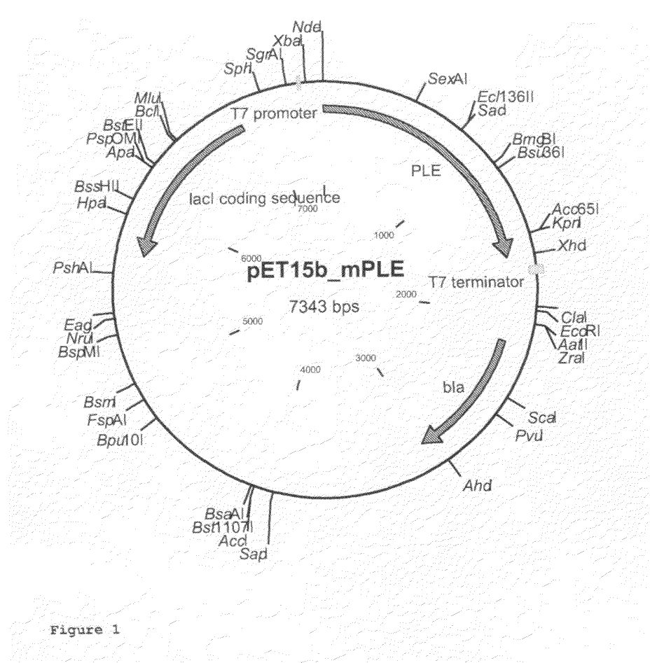

The sequence SEQ ID No. 1 coding for pig liver esterase (PLE) was amplified by PCR with primers 1 and 2 and, during this, an NdeI cleavage site was introduced at the 5′ end and an XhoI cleavage site at the 3′ end. The plasmid pCYTEX-PLE which was used in earlier studies on the cloning of pig liver esterase [Lange, S. et al., ChemBioChem (2001), 2, 576-582] was used as template. The PCR amplicon was treated with NdeI and XhoI and then introduced under standard conditions into the vector pET15b which had been treated in the same way. The construct obtained in this way, pET15bmPLE (see FIG. 1 and SEQ ID No. 2) was then transformed under standard conditions into the various E. coli expression strains.

### Comparative Example 1

### Expression of Recombinant PLE in Escherichia coli BL21Star™ (DE3)

The plasmid pET15bmPLE was transformed under standard conditions into E. coli BL21Star™. Single colonies were then cultured in 5 ml of LB medium mixed with ampicillin (100 μg/ml) at 30° C. overnight. The next day, the preculture was diluted in LB medium mixed with 100 μg/ml ampicillin to an OD₆₀₀ of 0.05 and then cultured at 30° C. and 200 rpm until the OD₆₀₀ was 1, and then expression was induced by adding IPTG to a final concentration of 100 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE to indicate expression in the cytosol; on the contrary, only inclusion bodies were detected. No activity was detectable in the activity assay.

### Comparative Example 2

### Expression of Recombinant PLE in Escherichia coli Rosetta (DE3)

The plasmid pET15bmPLE was transformed under standard conditions into E. coli Rosetta (DE3). Single colonies were then cultured in 5 ml of LB medium mixed with ampicillin (100 μg/ml) at 30° C. overnight. The next day, the preculture was diluted in LB medium mixed with 100 μg/ml ampicillin to an OD₆₀₀ of 0.05 and then cultured at 30° C. and 200 rpm until the OD₆₀₀ was 1, and then expression was induced by adding IPTG to a final concentration of 100 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE; on the contrary, only inclusion bodies were detected. No activity was detectable in the activity assay.

### Comparative Example 3

### Expression of Recombinant PLE in Escherichia coli Origami (DE3)

The plasmid pET15bmPLE was transformed under standard conditions into E. coli Origami (DE3). Single colonies were then cultured in 5 ml of LB medium mixed with ampicillin (100 μg/ml) at 30° C. overnight. The next day, the preculture was diluted in LB medium mixed with 100 μg/ml ampicillin to an OD₆₀₀ of 0.05 and then cultured at 30° C. and 200 rpm until the OD₆₀₀ was 1, and then expression was induced by adding IPTG to a final concentration of 100 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE; on the contrary, only inclusion bodies were detected. No activity was detectable in the activity assay.

### Comparative Example 4

### Expression of Recombinant PLE in Escherichia coli Rosetta-Gami B (DE3)

The plasmid pET15bmPLE was transformed under standard conditions into E. coli Rosetta-gami B (DE3). Single colonies were then cultured in 5 ml of LB medium mixed with ampicillin (100 μg/ml) at 30° C. overnight. The next day, the preculture was diluted in LB medium mixed with 100 μg/ml ampicillin to an OD₆₀₀ of 0.05 and then cultured at 30° C. and 200 rpm until the OD₆₀₀ was 1, and then expression was induced by adding IPTG to a final concentration of 100 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE; on the contrary, only inclusion bodies were detected. A small, scarcely quantifiable activity was detectable in the activity assay after 2 h, but was no longer detectable after 24 hours.

### Comparative Example 5

### Expression of Recombinant PLE in Escherichia coli Origami (DE3) with Addition of Ethanol to Induce Chaperones Belonging to E. coli

The plasmid pET15bmPLE was transformed under standard conditions into E. coli Origami (DE3). Single colonies were then cultured in 5 ml of LB medium mixed with ampicillin (100 μg/ml) at 30° C. overnight. The next day, the preculture was diluted in LB medium mixed with 100 μg/ml ampicillin and 3% (v/v) ethanol to induce the chaperones belonging to E. coli to an OD₆₀₀ of 0.05 and then cultured at 30° C. and 200 rpm until the OD₆₀₀ was 1, and then expression was induced by adding IPTG to a final concentration of 100 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE; on the contrary, only inclusion bodies were detected. No activity was detectable in the activity assay.

### Comparative Example 6

### Expression of recombinant PLE in Escherichia coli Origami (DE3) with Coexpression of the Chaperones Dnak, DnaJ, GrpE and GroEL, GroES

The plasmid pET15bmPLE was transformed under standard conditions into E. coli Origami (DE3) and then the plasmid pKJE7 was transformed into the strain resulting therefrom. Single colonies resulting therefrom were cultured in 5 ml of LB medium mixed with ampicillin (50 μg/ml) and chloramphenicol (20 μg/ml) at 30° C. overnight. The next day, the preculture was diluted in LB medium mixed with 100 μg/ml ampicillin and 50 μg/ml chloramphenicol to an OD₆₀₀ of 0.05. Immediately thereafter, expression of the chaperones Dnak, DnaJ, GrpE, GroEL and GroES was induced by adding arabinose to a final concentration of 1 mg/ml. Culturing was continued at 30° C. and 200 rpm until the OD₆₀₀ was 0.5, and expression of pig liver esterase was induced by adding IPTG to a final concentration of 40 μmol/l. 5 ml samples were taken after 2 and 24 hours and, after cell disruption with ultrasound, the samples were investigated by SDS-PAGE and assayed for activity with the activity assay described above. No soluble protein corresponding to PLE was detected in the SDS-PAGE; on the contrary, only inclusion bodies were detected. No activity was detectable in the activity assay.

Expression of Recombinant PLE in Escherichia coli Origami (DE3) with Coexpression of the Chaperones GroEL and GroES

The plasmid pET15bmPLE was transformed under standard conditions into E. coli Origami (DE3) and then the plasmid pGro7 was transformed into the strain resulting therefrom. Single colonies resulting therefrom were cultured in 5 ml of LB medium mixed with ampicillin (50 μg/ml) and chloramphenicol (20 μg/ml) at 30° C. overnight. The next day, the preculture was diluted in LB medium mixed with 100 μg/ml ampicillin and 50 μg/ml chloramphenicol to an OD₆₀₀ of 0.05. Immediately thereafter, expression of the chaperones GroEL and GroES was induced by adding arabinose to a final concentration of 1 mg/ml. Culturing was continued at 30° C. and 200 rpm until the OD₆₀₀ was 0.5, and expression of pig liver esterase was induced by adding IPTG to a final concentration of 40 μmol/l. 5 ml samples were taken after 2 and 24 hours and, after cell disruption with ultrasound, the samples were investigated by SDS-PAGE and assayed for activity with the activity assay described above. In the SDS-PAGE, a soluble protein corresponding to PLE was mainly detected, and no inclusion bodies were detectable. In the activity assay, an activity was detectable with the activity assay described above. It amounted to 9.94 U per ml of crude cell extract and the total protein content was 15.7 mg/ml determined by the Bradford method.

TABLE 1
Expression, inclusion body formation and
volumetric activities

| | | Activity* | |
|---|---|---|---|
| | PLE | volumetric | |
| E. coli strain | expression | [U/ml] | IB** |
| BL21Star ™ (DE3) (pET15bmPLE) | yes | 0 | yes |
| Rosetta (DE3) (pET15bmPLE) | yes | 0 | yes |
| Origami (DE3) (pET15bmPLE) | yes | 0 | yes |
| Rosetta-gami B (DE3) | yes | <0.2 | yes |
| (pET15bmPLE) | | | |
| Origami (DE3) (pET15bmPLE, | yes | 0 | yes |
| pKJE7) | | | |
| Origami (DE3) (pET15bmPLE, | yes | 0 | yes |
| pG-KJE8) | | | |
| Origami (DE3) (pET15bmPLE, | yes | 9.94 | n.d.*** |
| pGro7) | | | |

*Units based on the pNPA assay
**IB inclusion bodies
***n.d. not detectable

1-10. (canceled)

11. A microorganism comprising:
at least one copy of a polynucleotide sequence which is foreign to said microorganism and which encodes a protein having an enzymatic activity, and
a chaperone system which assists the functional expression of the protein in the form of an active enzyme.

12. The microorganism according to claim 11, wherein the microorganism is an E. coli strain.

13. The microorganism according to claim 11, wherein the polynucleotide sequence encodes an esterase.

14. The microorganism according to claim 13, wherein the polynucleotide sequence which encodes the esterase is a mammalian sequence, or a sequence which is homologous to a mammalian sequence and in which the codon usage is adapted to the host-specific codon usage.

15. The microorganism according to claim 14, wherein the sequence is a cDNA sequence from the pig genome or is a homologous sequence in which the codon usage has been adapted to the host-specific codon usage.

16. The microorganism according to claim 11, wherein the chaperone system includes the chaperones GroEL and GroES.

17. The microorganism according to claim 16, wherein the expression of the coding sequences for the chaperones GroEL and GroES is inducible.

18. The microorganism according to claim 16, wherein the organism is transgenic in relation to the coding sequences for the chaperones GroEL and GroES.

19. The microorganism according to claim 16, wherein the chaperone system does not include the chaperones Dnak, DnaJ and GrpE.

20. The microorganism according to claim 11, wherein the microorganism comprises the sequence SEQ ID NO: 1 or a homologous sequence.

21. The microorganism according to claim 11, wherein the microorganism comprises the sequence SEQ ID NO: 2.

22. A method for producing a protein having esterase activity, wherein the protein is expressed by a microorganism according to claim 11.

23. The method according to claim 22, further comprising purifying the protein after expression.

24. A method for producing a protein having esterase activity comprising culturing the microorganism of claim 11 for a time and under conditions suitable for expression of an active enzyme from said polynucleotide sequence.

25. The microorganism of claim 11, which encodes a stereoselective esterase.

26. The microorganism of claim 11, that comprises a polynucleotide sequence that encodes a stereoselective esterase comprising the amino acid sequence of SEQ ID NO: 5 or a fragment thereof; or an amino acid sequence homologous to SEQ ID NO: 5, or a fragment thereof.

27. A method for biocatalytic substrate conversion, comprising:
contacting a substrate with a biocatalyst comprising the microorganism of claim 11 or comprising a protein having an enzymatic activity encoded by the at least one polynucleotide sequence of the microorganism of claim 11.

28. The method of claim 27, wherein said biocatalyst is an esterase expressed by said microorganism which produces an optically active compound from the substrate.


SEQUENCE LISTING (5 sequences)

SEQ ID NO: 1 - DNA, 1638 bp, Sus
atggggcagccagcctcgccgcctgttgtggacactgcccagggccgagtcctggggaagtacgtcagcttagaaggcctggcacagccggtggccgtcttcctgggagtcccttttgccaagccccctctcggatccttgaggtttgctccgccgcagcctgcagaaccatggagcttcgtgaagaacaccacctcctaccctcccatgtgctgccaggacccagtagtggagcagatgacctcagatctatttaccaacggaaaggagaggctcactctggagttttctgaagactgtctctacctaaatatttacacccctgctgacctgacaaagaggggcagactgccggtgatggtgtggatccacggaggaggcctggtgttgggcggggcaccaatgtatgatggggtggtgcttgctgcgcatgaaaacgtggtggtggtggccatccagtaccgcctgggcatctggggattcttcagcacaggggatgaacacagccggggcaactggggtcacttggaccaggtggccgcactgcactgggtccaggagaacatcgccaactttggaggcgacccaggctctgtgaccatctttggagagtcagcaggaggggaaagtgtctctgttctggtgttgtctcccttggccaagaacctcttccaccgggccatctctgagagtggcgtggccctcactgttgccctggtcaggaaggacatgaaggctgcagctaagcaaattgctgtccttgctgggtgtaaaaccaccacctcggctgtctttgttcactgcctgcgccagaagtcggaggacgagctcttggacttaacgctgaagatgaaatttttaactcttgattttcatggagaccaaagagagagccatcccttcctgcccactgtggtggatggagtgctgctgcccaagatgcctgaagagattctggctgagaaggatttcaacactgtcccctacatcgtgggaatcaacaagcaagagtttggctggcttctgccaacgatgatgggcttccccctctctgaaggcaagctggaccagaagacggccacgtcactcctgtggaagtcctaccccatcgctaacatccctgaggaactgactccagtggccactgacaagtatttgggggggacagacgaccccgtcaaaaagaaagacctgttcctggacttgatgggggatgtggtgtttggtgtcccatctgtgacggtggcccgtcaacacagagatgcaggagcccccacctacatgtatgagtttcagtatcgcccaagcttctcatcggacaagaaacccaagacggtgatcggggaccacggggatgagatcttctccgtctttggttttccactgttaaaaggcgatgccccagaagaggaggtcagtctcagcaagacggtgatgaaattctgggccaactttgctcgcagtgggaaccccaatggggaggggctgccccattggccgatgtacgaccaggaagaagggtaccttcagatcggcgtcaacacccaggcagccaagaggctgaaaggtgaagaagtggccttctggaacgatctcctgtccaaggaggcagcaaagaagccacccaagataaagtga

SEQ ID NO: 2 - DNA, 7343 bp, Artificial Sequence
  DNA construct plasmid
tatggggcagccagcctcgccgcctgttgtggacactgcccagggccgagtcctggggaagtacgtcagcttagaaggcctggcacagccggtggccgtcttcctgggagtcccttttgccaagccccctctcggatccttgaggtttgctccgccgcagcctgcagaaccatggagcttcgtgaagaacaccacctcctaccctcccatgtgctgccaggacccagtagtggagcagatgacctcagatctatttaccaacggaaaggagaggctcactctggagttttctgaagactgtctctacctaaatatttacacccctgctgacctgacaaagaggggcagactgccggtgatggtgtggatccacggaggaggcctggtgttgggcggggcaccaatgtatgatggggtggtgcttgctgcgcatgaaaacgtggtggtggtggccatccagtaccgcctgggcatctggggattcttcagcacaggggatgaacacagccggggcaactggggtcacttggaccaggtggccgcactgcactgggtccaggagaacatcgccaactttggaggcgacccaggctctgtgaccatctttggagagtcagcaggaggggaaagtgtctctgttctggtgttgtctcccttggccaagaacctcttccaccgggccatctctgagagtggcgtggccctcactgttgccctggtcaggaaggacatgaaggctgcagctaagcaaattgctgtccttgctgggtgtaaaaccaccacctcggctgtctttgttcactgcctgcgccagaagtcggaggacgagctcttggacttaacgctgaagatgaaatttttaactcttgattttcatggagaccaaagagagagccatcccttcctgcccactgtggtggatggagtgctgctgcccaagatgcctgaagagattctggctgagaaggatttcaacactgtcccctacatcgtgggaatcaacaagcaagagtttggctggcttctgccaacgatgatgggcttccccctctctgaaggcaagctggaccagaagacggccacgtcactcctgtggaagtcctaccccatcgctaacatccctgaggaactgactccagtggccactgacaagtatttgggggggacagacgaccccgtcaaaaagaaagacctgttcctggacttgatgggggatgtggtgtttggtgtcccatctgtgacggtggcccgtcaacacagagatgcaggagcccccacctacatgtatgagtttcagtatcgcccaagcttctcatcggacaagaaacccaagacggtgatcggggaccacggggatgagatcttctccgtctttggttttccactgttaaaaggcgatgccccagaagaggaggtcagtctcagcaagacggtgatgaaattctgggccaactttgctcgcagtgggaaccccaatggggaggggctgccccattggccgatgtacgaccaggaagaagggtaccttcagatcggcgtcaacacccaggcagccaagaggctgaaaggtgaagaagtggccttctggaacgatctcctgtccaaggaggcagcaaagaagccacccaagataaagtgactcgaggatccggctgctaacaaagcccgaaaggaagctgagttggctgctgccaccgctgagcaataactagcataaccccttggggcctctaaacgggtcttgaggggttttttgctgaaaggaggaactatatccggatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagcca

SEQ ID NO: 3 - DNA, 30 bp, Artificial Sequence
  Synthetic DNA primer
gccatatggggcagccagcctcgccgcctg

SEQ ID NO: 4 - DNA, 29 bp, Artificial Sequence
  Synthetic DNA primer
gatcctcgagtcactttatcttgggtggc

SEQ ID NO: 5 - protein, 545 aa, Sus
MGQPASPPVVDTAQGRVLGKYVSLEGLAQPVAVFLGVPFAKPPLGSLRFAPPQPAEPWSFVKNTTSYPPMCCQDPVVEQMTSDLFTNGKERLTLEFSEDCLYLNIYTPADLTKRGRLPVMVWIHGGGLVLGGAPMYDGVVLAAHENVVVVAIQYRLGIWGFFSTGDEHSRGNWGHLDQVAALHWVQENIANFGGDPGSVTIFGESAGGESVSVLVLSPLAKNLFHRAISESGVALTVALVRKDMKAAAKQIAVLAGCKTTTSAVFVHCLRQKSEDELLDLTLKMKFLTLDFHGDQRESHPFLPTVVDGVLLPKMPEEILAEKDFNTVPYIVGINKQEFGWLLPTMMGFPLSEGKLDQKTATSLLWKSYPIANIPEELTPVATDKYLGGTDDPVKKKDLFLDLMGDVVFGVPSVTVARQHRDAGAPTYMYEFQYRPSFSSDKKPKTVIGDHGDEIFSVFGFPLLKGDAPEEEVSLSKTVMKFWANFARSGNPNGEGLPHWPMYDQEEGYLQIGVNTQAAKRLKGEEVAFWNDLLSKEAAKKPPKIK